(19) United States
(12) Patent Application Publication
(10) Pub. No.: US 2017/0202456 A1
(43) Pub. Date: Jul. 20, 2017

(54) MEASUREMENT APPARATUS

(71) Applicant: FUJI XEROX CO., LTD., Tokyo (JP)

(72) Inventors: Saori NISHIZAKI, Ebina-shi (JP); Hideo NAKAYAMA, Ebina-shi (JP); Taku KINOSHITA, Ebina-shi (JP); Kazutaka TAKEDA, Ebina-shi (JP); Hideaki OZAWA, Ebina-shi (JP); Kazuyuki MATSUSHITA, Ebina-shi (JP); Kohei YUKAWA, Ebina-shi (JP); Sho KIMURA, Ebina-shi (JP)

(73) Assignee: FUJI XEROX CO., LTD., Tokyo (JP)

(21) Appl. No.: 15/475,962

(22) Filed: Mar. 31, 2017

Related U.S. Application Data
(63) Continuation of application No. PCT/JP2015/082593, filed on Nov. 19, 2015.

(30) Foreign Application Priority Data
Nov. 26, 2014 (JP) 2014-239093

(51) Int. Cl. A61B 3/117

(52) U.S. Cl. CPC A61B 3/117

(57) ABSTRACT

A measurement apparatus includes: an eyelid opening section that comes into contact with skin on a periphery of an eyeball of a measurement subject and maintains an eyelid of the measurement subject in an open state; an emission section that emits light such that the light travels across an anterior chamber of the eyeball in which the eyelid is maintained in an open state by the eyelid opening portion; and a light reception section that receives the light which travels across the anterior chamber.

### CROSS REFERENCE TO RELATED APPLICATION

This is a continuation of International Application No. PCT/JP2015/082593 filed on Nov. 19, 2015, and claims priority from Japanese Patent Application No. 2014-239093, filed on Nov. 26, 2014.

### BACKGROUND

### Technical Field

The present invention relates to a measurement apparatus.

### SUMMARY

According to an aspect of the invention, there is provided a measurement apparatus includes: an eyelid opening section that comes into contact with skin on a periphery of an eyeball of a measurement subject and maintains an eyelid of the measurement subject in an open state; an emission section that emits light such that the light travels across an anterior chamber of the eyeball in which the eyelid is maintained in an open state by the eyelid opening portion; and a light reception section that receives the light which travels across the anterior chamber.

### BRIEF DESCRIPTION OF DRAWINGS

Exemplary embodiment(s) of the present invention will be described in detail based on the following figures, wherein:

FIG. 1 is a view illustrating an example of a configuration of an optical measurement apparatus in which the present exemplary embodiment is applied;

FIG. 2 is a view describing a relationship between an eyeball and an optical path of an optical system;

FIG. 3 is a view describing a method of measuring a rotation angle (optical rotation degree) of a polarization plane caused by an optically active substance contained in aqueous humor in an anterior chamber, by using the optical measurement apparatus;

FIGS. 4A, 4B, and 4C are views describing a configuration of an eyelid pressing section. Here, FIG. 4A is a perspective view of the optical measurement apparatus when viewed from the back side, FIG. 4B is a view describing a relationship between the eyelid pressing section and an eyelid, and FIG. 4C is a configuration diagram of an upper eyelid pressing section;

FIGS. 5A, 5B, and 5C are views describing an operation of the eyelid pressing section;

FIGS. 6A and 6B are views describing a configuration of an optical measurement apparatus in an alternative exemplary embodiment 1. Here, FIG. 6A is a perspective view of the optical measurement apparatus when viewed from the back side, and FIG. 6B is a cross-sectional view taken along arrow VIB in FIG. 6A;

FIGS. 7A, 7B, 7C, and 7D are views describing a configuration of an optical measurement apparatus in an alternative exemplary embodiment 2. Here, FIG. 7A is a perspective view of the optical measurement apparatus when viewed from the back side, FIG. 7B is a top view of an upper eyelid pressing section and a coupling member, FIG. 7C is a cross-sectional view taken along arrow VIIC in FIG. 7B, and FIG. 7D is a view describing an operation of the eyelid pressing section;

FIGS. 8A and 8B are views describing a configuration of an optical measurement apparatus in an alternative exemplary embodiment 3. Here, FIG. 8A is a perspective view of the optical measurement apparatus when viewed from the back side, and FIG. 8B is a cross-sectional view taken along arrow VIIIB in FIG. 8A; and

FIG. 9 is a view describing a configuration of an optical measurement apparatus in a modification example.

### DETAILED DESCRIPTION

Hereinafter, with reference to the accompanying drawings, exemplary embodiments of the present invention will be described.

(Optical Measurement Apparatus 1)

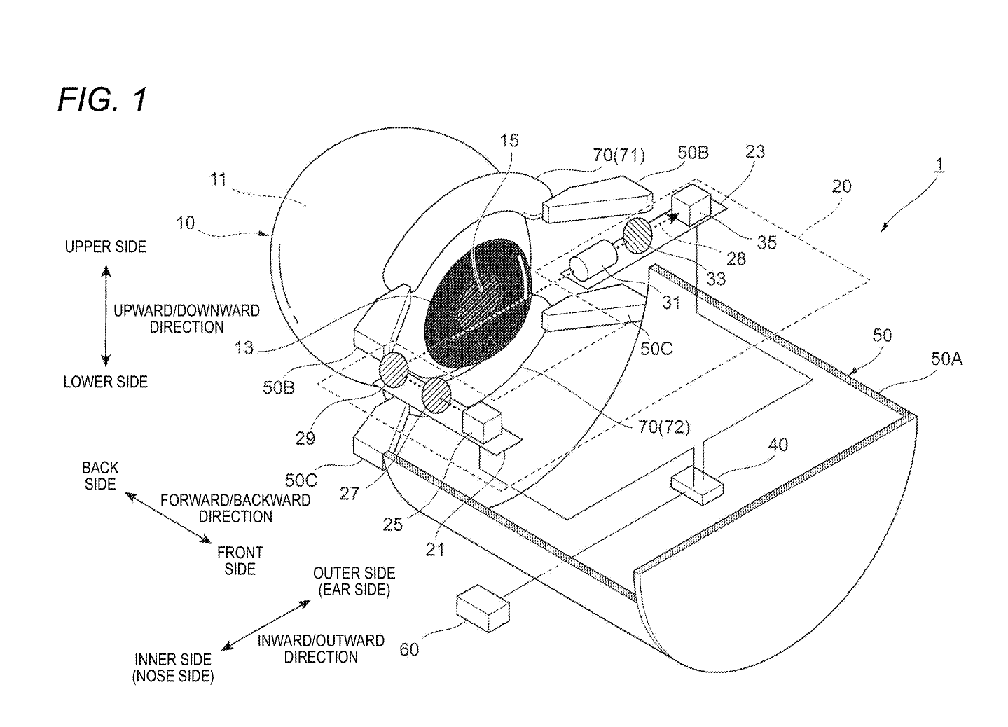

FIG. 1 is a view illustrating an example of a configuration of an optical measurement apparatus 1 in which the present exemplary embodiment is applied. The optical measurement apparatus 1 has a shape such that a measurement subject holds the optical measurement apparatus 1 in his/her own hand and can perform a measurement by wearing (applying) the optical measurement apparatus 1 in an eyeball by himself/herself. An eyeball 10 illustrated in FIG. 1 is a left eye.

The optical measurement apparatus (measurement apparatus) 1 includes an optical system 20 that is used for measuring characteristics of aqueous humor in an anterior chamber 13 (will be described later) of the eyeball 10 of a measurement subject, a control section 40 that controls the optical system 20, a holding section 50 that holds the optical system 20 and the control section 40, a calculation section 60 that calculates the characteristics of the aqueous humor based on data measured by using the optical system 20, and an eyelid pressing section 70 that comes into contact with an eyelid of the measurement subject and presses the eyelid.

In the description below, a direction crossing the upper side of the sheet and the lower side of the sheet regarding the optical measurement apparatus 1 illustrated in FIG. 1 is sometimes referred to as upward/downward direction. In addition, a direction crossing the front side of the measurement subject and the back side of the measurement subject illustrated in FIG. 1 is sometimes referred to as forward/backward direction. In addition, a direction crossing the inner side (nose side, inner canthus side) and the outer side (ear side, outer canthus side) when viewed from the measurement subject of the optical measurement apparatus 1 illustrated in FIG. 1 is sometimes referred to as inward/outward direction.

In addition, the characteristics of the aqueous humor measured by the optical measurement apparatus 1 in which the present exemplary embodiment is applied denotes a rotation angle (optical rotation degree α_{M}) of a polarization plane of linearly polarized light caused by an optically active substance contained in the aqueous humor, a color absorbance degree (circular dichroism) with respect to circularly polarized light, and the like. The polarization plane of linearly polarized light denotes a surface where the electric field of the linearly polarized light vibrates.

The optical system 20 includes a light emission system 21 that emits light with which the anterior chamber 13 (will be described later) of the eyeball 10 is irradiated, and a light reception system 23 that receives light which has passed through the anterior chamber 13.

First, the light emission system 21 which is an example of an emission portion includes a light emission portion 25, a polarizer 27, and a mirror 29.

The light emission portion 25 may be a light source having a wide wavelength width, such as a light emitting diode (LED) and a lamp or may be a light source having a narrow wavelength width such as a laser. It is preferable that the wavelength width is narrow. In addition, the light emission portion 25 may emit light having at least two or more wavelengths.

The polarizer 27 is a Nicol prism, for example. From rays of incident light, the polarizer 27 allows linearly polarized light having a predetermined polarization plane to pass through.

The mirror 29 causes an optical path 28 to be refracted. It is preferable that the mirror 29 maintains linearly polarized light without any change before and after reflection. It is not preferable that the mirror 29 disrupt the state of polarized light, such as rotating the polarization plane and causing the linearly polarized light to be elliptically polarized light. In a case where there is no need for the optical path 28 to be refracted, the mirror 29 may not be provided.

Subsequently, the light reception system 23 which is an example of a light reception portion includes a compensator 31, an analyzer 33, and a light reception portion 35.

For example, the compensator 31 is a magneto-optic element such as a Faraday element in which a garnet or the like is used. The compensator 31 rotates the polarization plane of linearly polarized light in response to a magnetic field.

The analyzer 33 is a member similar to the polarizer 27 and allows linearly polarized light having the predetermined polarization plane to pass through.

The light reception portion 35 is a light receiving element such as a silicon diode and outputs an output signal corresponding to the intensity of light.

The control section 40 controls the light emission portion 25, the compensator 31, the light reception portion 35, and the like in the optical system 20, thereby obtaining measurement data related to the characteristics of the aqueous humor.

The holding section 50 is an approximately cylindrical housing which holds the optical system 20 and the control section 40. In addition, the holding section 50 has a shape such that the measurement subject holds the holding section 50 in his/her own hand and may wear (apply) the holding section 50 in an eyeball of himself/herself. The holding section 50 illustrated in FIG. 1 exhibits a shape realized by cutting a cylinder along a plane parallel to an axial direction such that the optical system 20 is easily recognized. In addition, the shape of the holding section 50 may be a different shape. For example, a cross section may have a quadrangular or elliptic tube shape. In addition, in the cylindrical housing, the bottom surface on a side opposite to the side to be worn may be open or may be blocked by a different member.

The calculation section 60 receives measurement data from the control section 40 and calculates the characteristics of the aqueous humor.

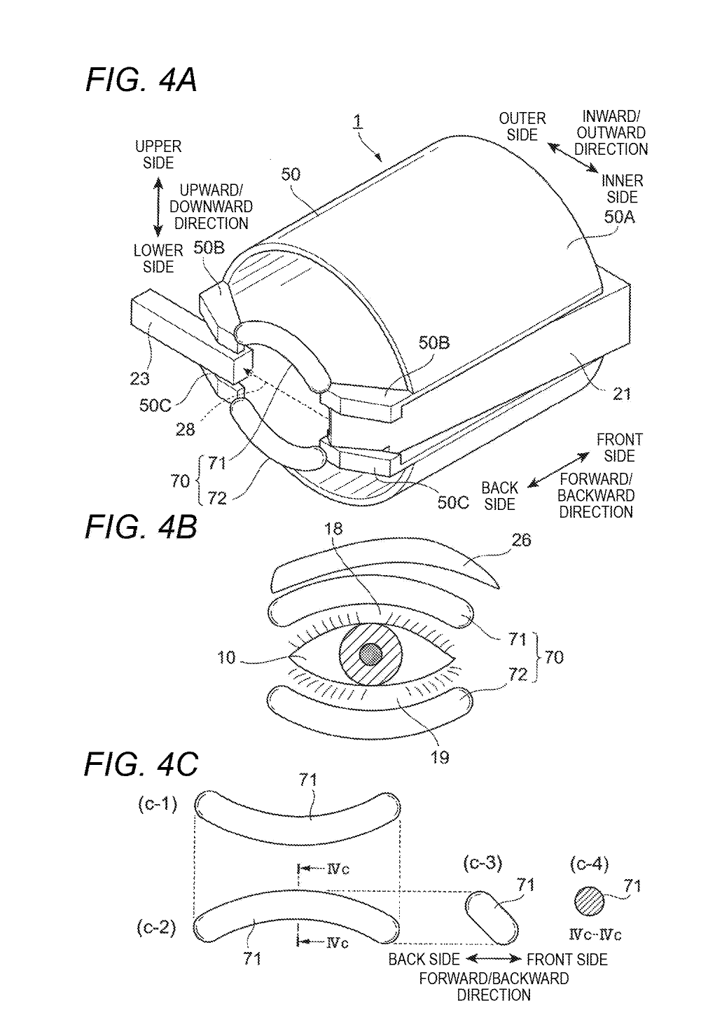

The eyelid pressing section 70 is provided in the holding section 50 and presses eyelids (upper eyelid 18 and lower eyelid 19, refer to FIG. 4B described below) by coming into contact with the eyelids, thereby maintaining the eyelids in an open state. The configuration of the eyelid pressing section 70 will be described later.

(Relationship between Eyeball 10 and Optical Path 28 of Optical System 20)

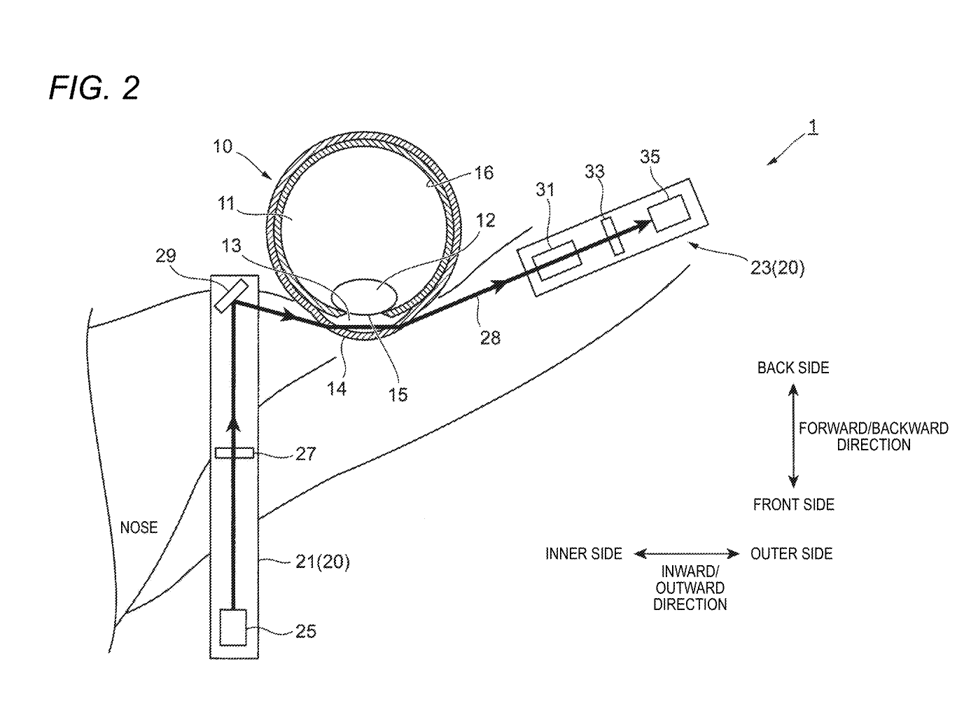

FIG. 2 is a view describing a relationship between the eyeball 10 and the optical path 28 of the optical system 20. FIG. 2 illustrates a state where a person (measurement subject) is viewed from the head side (upper side). In addition, in FIG. 2, a part of the optical system 20 appears to be embedded inside the face in relation to the uneven shape of the surface of the face. Actually, the optical system 20 is disposed on the surface of the face.

Subsequently, with reference to FIG. 2, a relationship between the eyeball 10 and the optical path 28 of the optical system 20 will be described.

Here, first, the structure of the eyeball 10 will be described. Subsequently, a relationship between the eyeball 10 and the optical path 28 of the optical system 20 will be described in detail.

As illustrated in FIG. 2, the eyeball 10 has a substantially spherical outer shape and a glass body 11 is present at the center. A crystalline lens 12 playing a role as a lens is embedded in a part of the glass body 11. The anterior chamber 13 is present on the front side of the crystalline lens 12, and a cornea 14 is present on the front side thereof. The peripheral portion of the crystalline lens 12 is surrounded by the iris, and the center thereof is a pupil 15. Excluding a portion being in contact with the crystalline lens 12, the glass body 11 is covered with a retina 16.

Here, the anterior chamber 13 is a region surrounded by the cornea 14 and the crystalline lens 12, and the anterior chamber 13 is a region bulging out from the spherical shape of the eyeball 10 in a convex shape. The anterior chamber 13 has a circular shape when viewed from the front. The anterior chamber 13 is filled with the aqueous humor.

Subsequently, a positional relationship between the eyeball 10 and the optical path 28 of the optical system 20 will be described.

As illustrated in FIG. 2, in the optical system 20, light used for measuring the characteristics of the aqueous humor is emitted from the light emission portion 25 and travels forward along the optical path 28, thereby being incident on the light reception portion 35. That is, light emitted from the light emission portion 25 passes through the polarizer 27. Thereafter, the light is refracted by the mirror 29 in a direction of traveling across the anterior chamber 13 (direction parallel to the eye). Then, the light passes through the direction of traveling across the anterior chamber 13 (inward/outward direction). Moreover, the light which has passed through the anterior chamber 13 is incident on the light reception portion 35 via the compensator 31 and the analyzer 33.

Here, as illustrated in FIG. 2, the light emitted from the light emission system 21 is incident on the anterior chamber 13 in an orientation toward the outer side (outer canthus side) in the inward/outward direction and in an orientation toward the front side in the forward/backward direction. In addition, the light which has passed through the anterior chamber 13 is incident on the light reception system 23 in the orientation toward the outer side in the inward/outward direction and in the orientation toward the back side in the forward/backward direction.

That is, the light emission system 21 (mirror 29) is disposed such that the light emitted toward the anterior chamber 13 by the light emission system 21 obliquely travels toward the front side in the forward/backward direction. In other words, the mirror 29 is disposed on the back side (inward side) with respect to an exposed portion (anterior chamber 13) of the eyeball 10 closer than the front side apex thereof.

In addition, the light reception system 23 (compensator 31) is disposed so as to receive light obliquely traveling from the anterior chamber 13 toward the back side in the forward/backward direction. In other words, the compensator 31 is disposed on the back side beyond the front side apex of the exposed portion (anterior chamber 13) of the eyeball 10.

The disposition is performed due to the following reason. That is, light emitted from the light emission portion 25 passes through the cornea 14 and is incident on the anterior chamber 13. In this case, since the refractive index (n=approximately 1.37) of the aqueous humor in the cornea 14 and the anterior chamber 13 is greater than that of air (n=approximately 1.0) and the anterior chamber 13 and the cornea 14 have convex shapes, the optical path 28 is refracted to the back side (eyeball 10 side). In addition, even after passing through the anterior chamber 13, the optical path 28 is further refracted to the back side. In consideration of the optical path 28 passing through the cornea 14 and the anterior chamber 13 and being refracted toward the back side, the light emission system 21 and the light reception system 23 are disposed.

In addition, the nose (bridge of the nose) is positioned around the eye (eyeball 10) in the face, and there is a small space for setting the optical system 20. Moreover, when light deviates from the anterior chamber 13, accurate measurements cannot be performed. Thus, it is preferable to set the optical path 28 such that light does not deviate from the anterior chamber 13 and the optical path 28 passes through the anterior chamber 13 so as to travel across the anterior chamber 13.

In addition, the optical path 28 is influenced by an optical path length which is the length of light passing through the aqueous humor in the anterior chamber 13. Therefore, as described above, it is favorable to set the optical path 28 such that the optical path length does not fluctuate. In the illustrated optical measurement apparatus 1, the optical path 28 is set so as to travel across the anterior chamber 13, an elongated optical path length may be set.

Furthermore, the eyeball 10 has a substantially spherical outer shape, and the nose (bridge of the nose) is positioned any of a side on which light is incident or a side on which passing light is received. Thus, there is a narrow space for the optical system 20 to be disposed.

In the illustrated optical measurement apparatus 1, the optical path 28 is set such that light is incident at an angle nearly parallel to the eyeball 10 and the optical path 28 travels across the anterior chamber 13. Therefore, as illustrated in FIG. 1, the space is intended to be effectively utilized by providing the mirror 29 and refracting the optical path 28 on the nose side. If the optical system 20 is a compact-type system, there is no need for the optical path 28 to be refracted.

The optical path 28 is not limited to the illustrated configuration and is favorable as long as the optical path 28 is set such that light emitted from the light emission portion 25 passes through the anterior chamber 13 so as to travel across the anterior chamber 13 and is received by the light reception portion 35. In addition, the circumstances where light passes through the anterior chamber 13 so as to travel across the anterior chamber 13 denote that the light passes through the anterior chamber 13 at an angle (that is, a range less than ±45 degrees with respect to a horizontal axis in the inward/outward direction) closer to the inward/outward direction than the upward/downward direction in a case where the eyeball 10 is viewed from the front, including a case where the light obliquely passes through the anterior chamber 13 in the forward/backward direction.

(Measurement of Aqueous Humor)

Subsequently, an example of measuring the aqueous humor in the anterior chamber 13 and calculating a glucose concentration of the aqueous humor by using the optical measurement apparatus 1 will be described.

The amount of injecting insulin to a diabetic patient is controlled depending on the glucose concentration in blood. Thus, it is required for the diabetic patient to grasp the glucose concentration in blood at all times. As a method of measuring the glucose concentration in blood, there is a method in which a fingertip or the like is punctured with an injection needle and a very small quantity of blood is gathered. However, in this method, even in a case of a very small quantity of blood, the diabetic patient feels pain when collecting blood, thereby accompanying a mental burden. Accordingly, there is a high demand for a noninvasive-type test method replacing an invasive-type test method such as puncturing.

Here, the aqueous humor in the anterior chamber 13 having substantially the same component as that of blood serum contains protein, glucose, ascorbic acid, and the like. It is known that there is a correlationship between the glucose concentration in blood and the glucose concentration in the aqueous humor. Moreover, in the aqueous humor, generally, there is no cell substance of blood, and there is small influence of light scattering. Protein, glucose, ascorbic acid, and the like contained in the aqueous humor are the optically active substances and have optical activities.

In the optical measurement apparatus 1 in which the present exemplary embodiment is applied, while the aqueous humor is utilized, the concentration of glucose or the like having the optical activities is optically measured.

(Setting Optical Path)

In a technique of optically measuring the concentration or the like of the optically active substances such as glucose contained in the aqueous humor, two optical paths can be set as follows.

In one optical path being different from the configuration illustrated in FIG. 2, light is incident at an angle nearly perpendicular to the eyeball 10, that is, along the forward/backward direction, the light is reflected by the interface between the cornea 14 and the aqueous humor or the interface between the aqueous humor and the crystalline lens 12, and the reflected light is received (detected). In the other optical path as in the configuration illustrated in FIG. 2, light is incident at an angle intersecting the forward/backward direction, specifically at an angle nearly parallel to the eyeball 10, and the light which has passed through the aqueous humor in the anterior chamber 13 is received (detected).

In an optical path such as the former above in which light is incident at an angle nearly perpendicular to the eyeball 10, there is a possibility that the light reaches the retina 16. Particularly, in a case of using a laser having high coherency in the light emission portion 25, it is not preferable when light reaches the retina 16.

In contrast, in an optical path such as the latter above in which light is incident at an angle nearly parallel to the eyeball 10, the light passes through the anterior chamber 13 so as to travel across the anterior chamber 13 via the cornea 14, and the light which has passed through the aqueous humor is received (detected). Therefore, the light is restrained from reaching the retina 16.

(Calculation of Concentration of Optically Active Substance)

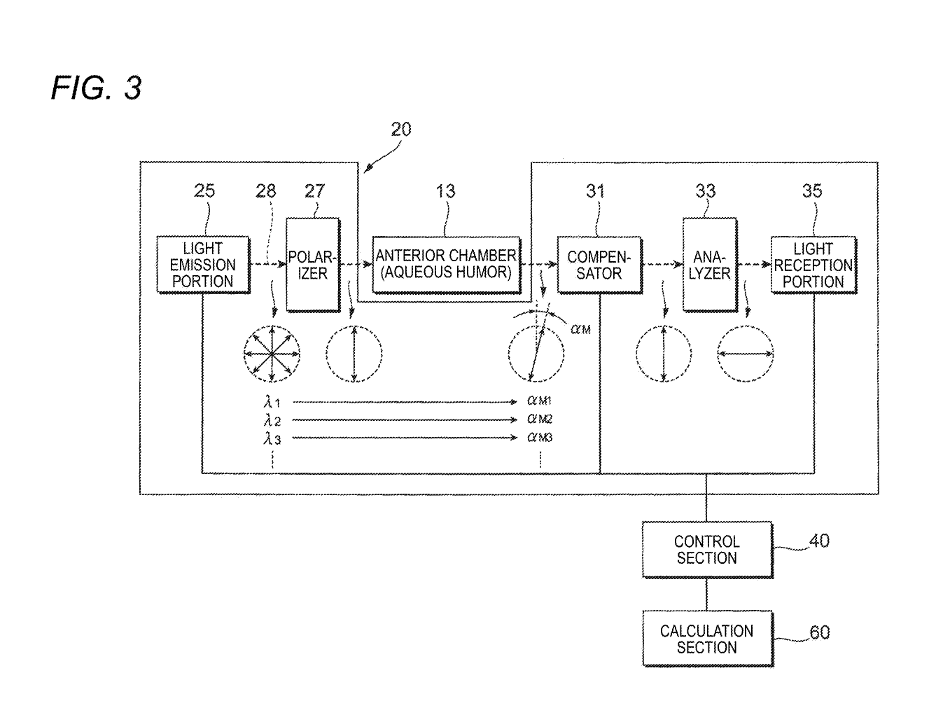

FIG. 3 is a view describing a method of measuring the rotation angle (optical rotation degree) of the polarization plane caused by the optically active substance contained in the aqueous humor in the anterior chamber 13, by using the optical measurement apparatus 1. Here, in order to make description easy, the optical path 28 is configured not to be refracted (to be linear), and illustration of the mirror 29 is omitted.

In addition, in each of the spaces among the light emission portion 25, the polarizer 27, the anterior chamber 13, the compensator 31, the analyzer 33, and the light reception portion 35 illustrated in FIG. 3, the states of polarized light viewed in traveling directions of the light are respectively indicated with arrows in a circle.

The light emission portion 25 emits light having a random polarization plane. The polarizer 27 allows linearly polarized light having the predetermined polarization plane to pass through. In FIG. 3, as an example, linearly polarized light having the polarization plane parallel to the sheet passes through.

The polarization plane of the linearly polarized light which has passed through the polarizer 27 is rotated by the optically active substance contained in the aqueous humor in the anterior chamber 13. In FIG. 3, the polarization plane rotates by the angle α_{M} (optical rotation degree α_{M}).

Subsequently, a magnetic field is applied to the compensator 31 such that the polarization plane rotated due to the optically active substance contained in the aqueous humor in the anterior chamber 13 returns to the original state.

The linearly polarized light which has passed through the analyzer 33 is received by the light reception portion 35 and is converted into an output signal corresponding to the intensity of light.

Here, an example of the method of measuring the optical rotation degree α_{M} by using the optical system 20 will be described.

First, in a state where light emitted from the light emission portion 25 is prohibited from passing through the anterior chamber 13, while the optical system 20 including the light emission portion 25, the polarizer 27, the compensator 31, the analyzer 33, and the light reception portion 35 is used, the compensator 31 and the analyzer 33 are set such that an output signal of the light reception portion 35 is minimized. In the example illustrated in FIG. 3, in a state where light is prohibited from passing through the anterior chamber 13, the polarization plane of the linearly polarized light which has passed through the polarizer 27 becomes orthogonal to the polarization plane passing through the analyzer 33.

Subsequently, a state where light passes through the anterior chamber 13 is established. Then, the polarization plane rotates due to the optically active substance contained in the aqueous humor in the anterior chamber 13. Therefore, the output signal from the light reception portion 35 deviates from the minimum value. A magnetic field to be applied to the compensator 31 is set such that the output signal from the light reception portion 35 is minimized That is, the polarization plane is rotated by the compensator 31 so as to be orthogonal to the polarization plane passing through the analyzer 33.

The angle of the polarization plane rotated by the compensator 31 corresponds to the optical rotation degree α_{M} caused by the optically active substance contained in the aqueous humor. Here, the relationship between the magnitude of the magnetic field applied to the compensator 31 and the angle of the rotated polarization plane is known in advance. Therefore, based on the magnitude of the magnetic field applied to the compensator 31, the optical rotation degree α_{M} is ascertained.

Specifically, rays of light having plural wavelengths λ (wavelengths λ₁, λ₂, λ₃, and so on) are incident on the aqueous humor in the anterior chamber 13 from the light emission portion 25, and the optical rotation degrees α_{M} (optical rotation degrees α_{M1}, α_{M2}, α_{M3}, and so on) are respectively obtained with respect to the wavelengths. The sets of the wavelength λ and the optical rotation degree α_{M} are taken into the calculation section 60, and the concentration of an intended optically active substance is calculated.

The concentration of the optically active substance calculated by the calculation section 60 may be displayed through a display section (not illustrated) included in the optical measurement apparatus 1 or may be output to a different terminal device (not illustrated) such as a personal computer (PC) via an output section (not illustrated) included in the optical measurement apparatus 1.

Furthermore, as described above, the aqueous humor contains plural optically active substances. Thus, the measured optical rotation degree α_{M} is the sum of each of the optical rotation degrees α_{M} of the plural optically active substances. Therefore, the concentration of the intended optically active substance is required to be calculated from the measured optical rotation degree α_{M}. For example, the concentration of the intended optically active substance can be calculated by using a known method such as that disclosed in JP-A-09-138231. Thus, description will be omitted herein.

In addition, in FIG. 3, both the polarization plane of the polarizer 27 and the polarization plane before passing through the analyzer 33 are parallel to the sheet. However, in a case where the polarization plane is rotated by the compensator 31 in advance, the polarization plane before passing through the analyzer 33 may incline from a plane parallel to the sheet. That is, in a state where light does not pass through the aqueous humor in the anterior chamber 13, it is favorable to set the compensator 31 and the analyzer 33 such that the output signal of the light reception portion 35 is minimized

In addition, here, an example of using the compensator 31 is described as a method of obtaining the optical rotation degree α_{M}. However, the optical rotation degree α_{M} may be obtained by using a portion other than the compensator 31. Moreover, here, an orthogonal polarizer method (however, the compensator 31 is used) which is the most basic measurement method of measuring the rotation angle (optical rotation degree α_{M}) of the polarization plane is described. However, other measurement methods such as a rotation analyzer method, a Faraday modulation method, and an optical delay modulation method may be applied.

(Structure of Eyelid Pressing Section 70)

FIGS. 4A, 4B, and 4C are views describing a configuration of the eyelid pressing section 70. Specifically, FIG. 4A is a perspective view of the optical measurement apparatus 1 when viewed from the back side, FIG. 4B is a view describing a relationship between the eyelid pressing section 70 and the eyelid, and FIG. 4C is a configuration diagram of an upper eyelid pressing section 71. In FIG. 4C, (c-1) is a top view of the upper eyelid pressing section 71, (c-2) is a front view of the upper eyelid pressing section 71, (c-3) is a side view of the upper eyelid pressing section 71, and (c-4) is a cross-sectional view taken along line IVC-IVC in (c-2).

Subsequently, with reference to FIGS. 4A, 4B, and 4C, the eyelid pressing section 70 which is an example of an eyelid opening portion will be described.

First, when light which has passed through the aqueous humor is detected and measures the concentration of glucose or the like by using the optical measurement apparatus 1, in order to restrain the influence of eyelids, eyelashes, or the like which can be hindrance to the optical path, it is preferable that an eye (eyelid) of the measurement subject is maintained in an open state.

As illustrated in FIG. 4A, the optical measurement apparatus 1 in the present exemplary embodiment includes the eyelid pressing section 70 pressing the eyelids of the measurement subject. The eyelid pressing section 70 is provided at the end portion of the holding section 50 on the back side. In the illustrated example, the eyelid pressing section 70 includes the upper eyelid pressing section 71 and a lower eyelid pressing section 72. In addition, in the optical measurement apparatus 1, parts which protrude the most to the back side are the upper eyelid pressing section 71 and the lower eyelid pressing section 72.

The upper eyelid pressing section 71 and the lower eyelid pressing section 72 are respectively disposed on the upper side and the lower side beyond the light emission system 21 and the light reception system 23. In other words, the upper eyelid pressing section 71 and the lower eyelid pressing section 72 are provided face to face while interposing the optical path 28 therebetween.

In the illustrated example, the upper eyelid pressing section 71 and the lower eyelid pressing section 72 are supported by the holding section 50. Specifically, the upper eyelid pressing section 71 and the lower eyelid pressing section 72 are fixedly provided at the end portion of a cylindrical main body 50A on the back side and are respectively supported by an upper support portion 50B and a lower support portion 50C extending along the optical path 28.

Furthermore, if the upper eyelid pressing section 71, the lower eyelid pressing section 72, the light emission system 21, and the light reception system 23 are intended to be individually disposed as separate bodies in a limited space on the periphery of the eyeball 10 where the nose, the eyelashes, and the like are present, interference is likely to occur among the members. Therefore, when the members are integrally supported by the holding section 50, each of the members is easily disposed in the limited space.

As illustrated in FIG. 4B, the upper eyelid pressing section 71 and the lower eyelid pressing section 72 are provided at positions respectively face to face with the upper eyelid 18 and the lower eyelid 19 in the holding section 50. When the upper eyelid pressing section 71 and the lower eyelid pressing section 72 are pressed against the upper eyelid 18 and the lower eyelid 19, the upper eyelid 18 and the lower eyelid 19 are in a state where movement thereof is restricted. That is, the upper eyelid pressing section 71 and the lower eyelid pressing section 72 also function as restriction portions which restrict movement of the upper eyelid 18 and the lower eyelid 19.

Subsequently, by using FIG. 4C, the shapes of the upper eyelid pressing section 71 and the lower eyelid pressing section 72 will be described. Here, description is given by using the upper eyelid pressing section 71. The upper eyelid pressing section 71 and the lower eyelid pressing section 72 are symmetric to each other while having a plane as the reference in which the upward/downward direction indicates the normal line (refer to FIG. 4A).

As illustrated in FIG. 4C, the upper eyelid pressing section 71 is a rod-like member (substantially columnar member) having a circularly-shaped cross section (refer to (c-4) in FIG. 4C). In addition, the outer circumferential surface of the upper eyelid pressing section 71 which comes into contact with the eyelids is formed so as to have a smoothly continuous curve surface, and no corner portion is formed on the outer circumferential surface of the upper eyelid pressing section 71 which comes into contact with the eyelids.

The upper eyelid pressing section 71 is provided so as to have a shape along the upper eyelid 18 (refer to FIG. 4B), that is, so as to be curved along the eyeball 10 (FIG. 4B). Specifically, as illustrated in (c-1) to (c-3) in FIG. 4C, a central portion in the longitudinal direction is curved in the orientation protruding to the front side and in the orientation protruding to the upper side. As illustrated in FIG. 4A, the upper eyelid pressing section 71 and the lower eyelid pressing section 72 are disposed such that the central sides thereof in the inward/outward direction are curved in the orientation of being separated from each other.

For example, the eyelid pressing section 70 (upper eyelid pressing section 71 and lower eyelid pressing section 72) are formed of a silicone resin (silicone). The eyelid pressing section 70 may be configured of a different resin, metal, or the like. For example, the eyelid pressing section 70 may be formed by applying an acrylic pressure sensitive adhesive to a resin formed of vinyl chloride or the like. Specifically, for example, the eyelid pressing section 70 may have a configuration in which a medical adhesive tape is provided on the outer circumferential surface of the eyelid pressing section 70. Furthermore, it is preferable that a material having high frictional force and a high level of safety is used for the eyelid pressing section 70. As described below, in a case where the eyelids are respectively squeezed upward and downward in the inward direction of the eyeball 10 along the eyeball 10, the eyelid pressing section 70 may be configured with an elastic member which is deformed so as to squeeze the eyelid to the inward side while being open toward the inward direction along the eyeball 10 in a case where the eyelid pressing section 70 is pressed against the eyelids. When such a configuration is employed, the eyelids are respectively squeezed upward and downward in the inward direction of the eyeball 10 along the eyeball 10.

(Operation of Eyelid Pressing Section 70)

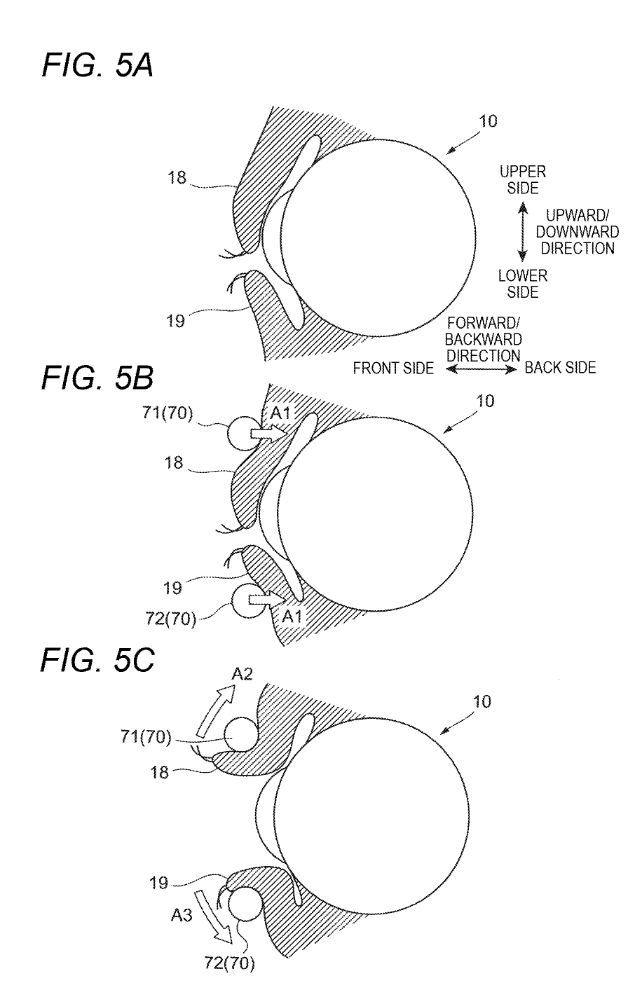

FIGS. 5A, 5B, and 5C are views describing an operation of the eyelid pressing section 70. FIGS. 5A, 5B, and 5C illustrate a state where the periphery of the eyeball 10 is viewed in the orientation from the inner side (nose side) toward the outer side (ear side).

Subsequently, with reference to FIGS. 5A, 5B, and 5C, the operation of the eyelid pressing section 70 will be described.

When the eyelid pressing section 70 (upper eyelid pressing section 71 and lower eyelid pressing section 72) having the above-described configuration is pressed against the eyelids (upper eyelid 18 and lower eyelid 19) of the measurement subject, a state where a gap is formed between the upper eyelid 18 and the lower eyelid 19, that is, an open state of the eyelids is maintained. Accordingly, the optical path 28 passing through the aqueous humor of the anterior chamber 13 is ensured (refer to FIGS. 4A 4B, and 4C).

Hereinafter, specific description will be given.

First, FIG. 5A illustrates a state of the eyelids (upper eyelid 18 and lower eyelid 19) before the measurement subject wears the optical measurement apparatus 1 (refer to FIG. 1), that is, before the eyelid pressing section 70 (upper eyelid pressing section 71 and lower eyelid pressing section 72) is pressed against the eyelids. In this state, the upper eyelid 18 and the lower eyelid 19 may freely move (be freely open and closed).

Subsequently, as illustrated in FIG. 5B, the measurement subject wears the optical measurement apparatus 1 in the eyelids (refer to FIG. 1). That is, the upper eyelid pressing section 71 and the lower eyelid pressing section 72 come into contact with the eyelids (upper eyelid 18 and lower eyelid 19). Accordingly, movement of each of the upper eyelid 18 and the lower eyelid 19 is restricted. From this state, the optical measurement apparatus 1 is further pressed against the eyelids. That is, the upper eyelid pressing section 71 and the lower eyelid pressing section 72 are moved to the back side (in the inward direction with respect to the eyeball) (refer to arrow A1).

Then, as illustrated in FIG. 5C, the upper eyelid 18 and the lower eyelid 19 move along the eyeball 10 (refer to arrows A2 and A3), and the eyelids are in an open state. More specifically, the upper eyelid pressing section 71 and the lower eyelid pressing section 72 move upward and downward in the inward direction of the eyeball 10 due to pressing in accordance with positioning of the light emission system 21 and the light reception system 23. Accordingly, the upper eyelid 18 and the lower eyelid 19 are squeezed toward the inward direction of the eyeball 10, and thus, the eyelids are open.

Here, the holding section 50 integrally holds the eyelid pressing section 70, the light emission system 21, and the light reception system 23. When positioning is performed, in accordance with the eyelid pressing section 70 being pressed toward the back side of the eyeball 10, the light emission system 21 and the light reception system 23 also move toward the back side at the same time. The eyelid pressing section 70 or the holding section 50 abuts on a site on the periphery of the eyeball (forehead, cheek, or the like) of the measurement subject, and the optical measurement apparatus 1 is positioned at a position from which the optical measurement apparatus 1 no longer moves to the back side. That is, in a state where the holding section 50 is positioned and the eyelids are open, the holding section 50 holds the light emission system 21 and the light reception system 23 at positions in which light emitted from the light emission system 21 travels across the anterior chamber of the eyeball and the light traveling across is received by the light reception system 23. According to such a configuration, opening of the eyelids and positioning of the light emission system 21 and the light reception system 23 are performed at the same time. In a state where the eyelids are open, light passes through the aqueous humor, thereby measuring the glucose concentration or the like.

After the measurement ends, when the optical measurement apparatus 1 (refer to FIG. 1) is detached from the measurement subject, the upper eyelid pressing section 71 and the lower eyelid pressing section 72 are separated from the eyelids (upper eyelid 18 and lower eyelid 19), and movement of the upper eyelid 18 and the lower eyelid 19 is no longer restricted.

In addition, being different from the illustrated example, in a state where the eyelids (upper eyelid 18 and lower eyelid 19) are open wider than those in the state illustrated in FIG. 5A, in other words, in an open state where the eye is normally open, there are cases where the measurement subject wears the optical measurement apparatus 1 (refer to FIG. 1). In this case as well, since the upper eyelid pressing section 71 and the lower eyelid pressing section 72 respectively come into contact with the upper eyelid 18 and the lower eyelid 19, and the upper eyelid 18 and the lower eyelid 19 are in a state where movement thereof is restricted, the eyelids are maintained in an open state. In FIGS. 5A, 5B, and 5C, the positions of the upper eyelid pressing section 71 and the lower eyelid pressing section 72 and the shapes of the eyelids are illustrated such that the features of Example are described so as to be easily understood. FIGS. 5A, 5B, and 5C are not necessarily illustrating the actual positions of the members and the actual state of the eyelids.

In addition, in the present exemplary embodiment, description is given regarding the configuration in which the upper eyelid 18 and the lower eyelid 19 are squeezed toward the inward direction of the eyeball 10. However, as long as a configuration in which movement is restricted due to being in contact with the upper eyelid 18 and the lower eyelid 19, the configuration of being squeezed may not be applied. Moreover, a configuration in which movement of the upper eyelid 18 and the lower eyelid 19 is restricted due to being in contact with the periphery of the eyeball 10 of the measurement subject other than the upper eyelid 18 and the lower eyelid 19 may be applied.

In addition, in the present exemplary embodiment, as in the exemplary embodiment described below, a configuration in which the upper eyelid 18 and the lower eyelid 19 are squeezed toward the inward direction of the eyeball 10 by using a driving source such as a motor so as to move the upper eyelid pressing section 71 and the lower eyelid pressing section 72 upward and downward in the inward direction of the eyeball 10 may be applied.

### Alternative Exemplary Embodiment 1

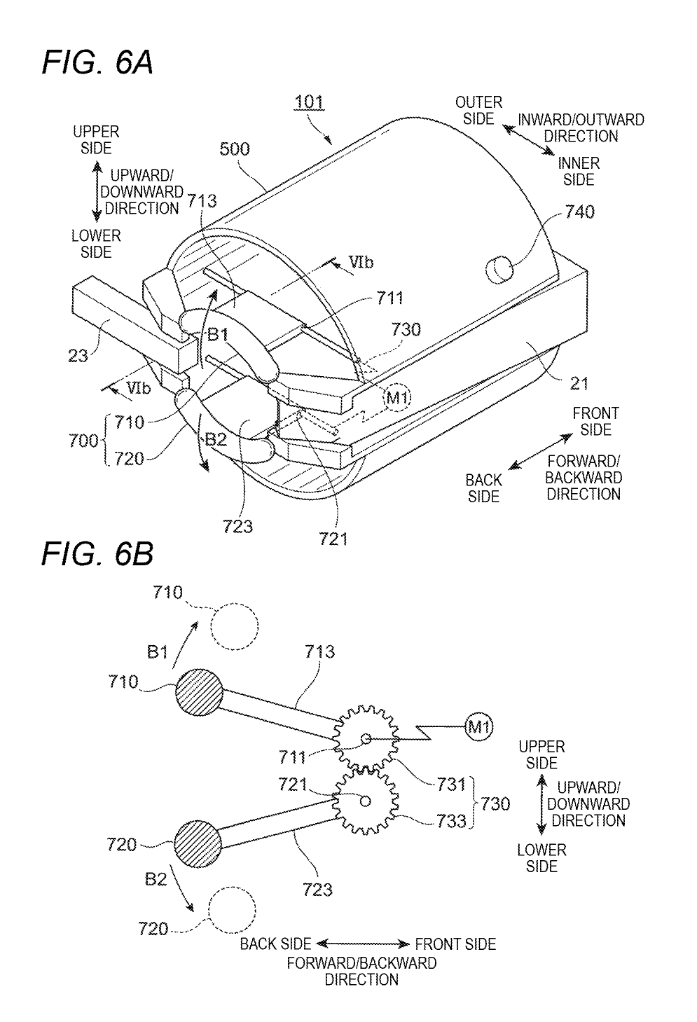

FIGS. 6A and 6B are views describing a configuration of an optical measurement apparatus 101 in an alternative exemplary embodiment 1. Specifically, FIG. 6A is a perspective view of the optical measurement apparatus 101 when viewed from the back side, and FIG. 6B is a cross-sectional view taken along arrow VIB in FIG. 6A.

Subsequently, with reference to FIGS. 6A and 6B, the alternative exemplary embodiment 1 will be described.

In the above-described optical measurement apparatus 1 illustrated in FIG. 1 and the like, description is given regarding the configuration in which the position of the eyelid pressing section 70 is fixed. However, the configuration is not limited thereto. For example, as in the optical measurement apparatus 101 illustrated in FIG. 6A, an eyelid pressing section 700 (upper eyelid pressing section 710 and lower eyelid pressing section 720) may be movably provided.

Specifically, the optical measurement apparatus 101 illustrated in FIG. 6A includes a motor (driving source) M1, a gear group (transmitting portion) 730 that transmits driving force from the motor M1, rotary axes 711 and 721 that extend along the inward/outward direction, and coupling members (support portions) 713 and 723 that are respectively connected to the upper eyelid pressing section 710 and the lower eyelid pressing section 720. The motor M1, the gear group 730, the rotary axes 711 and 721, and the coupling members 713 and 723 are provided on the inner side of a holding section 500. In addition, the optical measurement apparatus 101 includes an operation button 740 that triggers driving of the motor M1.

Here, as illustrated in FIG. 6B, the gear group 730 includes a gear 731 and a gear 733 that rotate upon a reception of driving from the motor M1. The gear 731 and the gear 733 respectively rotate around the rotary axes 711 and 721.

In addition, as illustrated in FIG. 6B, in the coupling members 713 and 723, the gear 731 and the gear 733 are respectively connected to one ends thereof on the front side, and the upper eyelid pressing section 710 and the lower eyelid pressing section 720 are respectively connected to the other ends thereof on the back side.

An operation of the optical measurement apparatus 101 will be described. First, the measurement subject wears the optical measurement apparatus 101 in the eyelids, and the upper eyelid pressing section 710 and the lower eyelid pressing section 720 come into contact with the upper eyelid 18 and the lower eyelid 19 (refer to FIG. 4B).

In this state, for example, when the measurement subject operates the operation button 740, the motor M1 is driven. In response to the driving of the motor M1, the upper eyelid pressing section 710 and the lower eyelid pressing section 720 move in the orientation of being separated from each other, via the gears 731 and 733 and the coupling members 713 and 723 (refer to arrows B1 and B2). Accordingly, the upper eyelid 18 and the lower eyelid 19 are open. In this manner, in a state where the upper eyelid 18 and the lower eyelid 19 are open, the optical measurement apparatus 101 is in a positioned state with respect to the eyeball 10.

Here, the holding section 500 holds the light emission system 21 and the light reception system 23 at positions in which light emitted from the light emission system 21 travels across the anterior chamber of the eyeball 10 in a state where the upper eyelid 18 and the lower eyelid 19 are open, and the light traveling across is received by the light reception system 23. Thus, when a measurement is performed, the optical path traveling across the anterior chamber is ensured in state where the upper eyelid 18 and the lower eyelid 19 are open.

For example, a part of the holding section 500 abuts on a site on the periphery of the eyeball (forehead, cheek, or the like) of the measurement subject, and positioning of the optical measurement apparatus 101 in the forward/backward direction is performed. In this state, the upper eyelid pressing section 710 and the lower eyelid pressing section 720 may be configured to be in a state of being in contact with the upper eyelid 18 and the lower eyelid 19.

That is, the holding section 500 may include an abutment portion which abuts on a site on the periphery of the eyeball of the measurement subject, and positioning of the light emission system 21 and the light reception system 23 in the forward/backward direction may be performed by using the abutment portion. The upper eyelid pressing section 710 and the lower eyelid pressing section 720 may be brought into contact with the upper eyelid 18 and the lower eyelid 19 through the positioning operation, and thereafter, the upper eyelid 18 and the lower eyelid 19 may be open in accordance with driving of the motor M1.

In other words, the upper eyelid pressing section 710 and the lower eyelid pressing section 720 are provided at positions where the light emission system 21 and the light reception system 23 come into contact with skin on the periphery of the eyeball 10 through the positioning operation of the light emission system 21 and the light reception system 23 with respect to the eyeball 10, that is, at positions where in a state where the light emission system 21 and the light reception system 23 are positioned with respect to the eyeball 10, the upper eyelid 18 and the lower eyelid 19 can be open and closed.

When the light emission system 21, the light reception system 23, the upper eyelid pressing section 710, and the lower eyelid pressing section 720 are held by the holding section 500 in such a positional relationship, positioning of the light emission system 21 and the light reception system 23 with respect to the eyeball 10 and positioning of the upper eyelid pressing section 710 and the lower eyelid pressing section 720 with respect to the upper eyelid 18 and the lower eyelid 19 are performed at the same time.

In addition, when a mechanism of driving the upper eyelid pressing section 710 and the lower eyelid pressing section 720 is used, regardless of the positioning operation of the light emission system 21 and the light reception system 23 with respect to the eyeball 10. an opening and closing operation of the upper eyelid pressing section 710 and the lower eyelid pressing section 720 may be performed. Therefore, misalignment of the light emission system 21 and the light reception system 23 through the opening and closing operation is unlikely to occur.

Moreover, since positioning of the light emission system 21 and the light reception system 23 in the forward/backward direction is not performed through the abutment of the upper eyelid pressing section 710 and the lower eyelid pressing section 720 and the abutment portion of the holding section 500 is positioned by abutting on a site on the periphery of the eyeball of the measurement subject, even in a positioned state, the opening and closing operation of the upper eyelid pressing section 710 and the lower eyelid pressing section 720 is easily performed.

In the holding section 500 in FIG. 6A, regions of the holding section 500 positioned on both sides of the upper eyelid pressing section 710 and the lower eyelid pressing section 720 correspond to the abutment portions. However, the abutment portion is not limited thereto. As necessary, an abutment member may be provided so as to protrude from a tabularly-shaped holding section 500. In addition, the abutment position is not limited to the periphery of the eyeball. The abutment position may be an arbitrary position on a facial surface of the measurement subject.

In addition, in the optical measurement apparatus 101, the eyelids can be more reliably open by driving the motor M1.

Furthermore, for example, even in a case where the measurement subject wears the optical measurement apparatus 101 in a state of closing the eyelids, the eyelids are open by the optical measurement apparatus 101 after wearing the optical measurement apparatus 101. Accordingly, psychologically anxiety which the measurement subject can have when wearing the optical measurement apparatus 101 is reduced.

As a modification example of the present exemplary embodiment, the positional relationship between the abutment portion, and the upper eyelid pressing section 710 and the lower eyelid pressing section 720 in the holding section 500 may be utilized such that movement of the upper eyelid 18 and the lower eyelid 19 is restricted by the upper eyelid pressing section 710 and the lower eyelid pressing section 720 being in contact therewith. The upper eyelid 18 and the lower eyelid 19 are maintained in an open state, and in such a state, a measurement may be performed. That is, without having any driving source or the like, the upper eyelid 18 and the lower eyelid 19 may be maintained in an open state by the upper eyelid pressing section 710 and the lower eyelid pressing section 720 which are not driven.

### Alternative Exemplary Embodiment 2

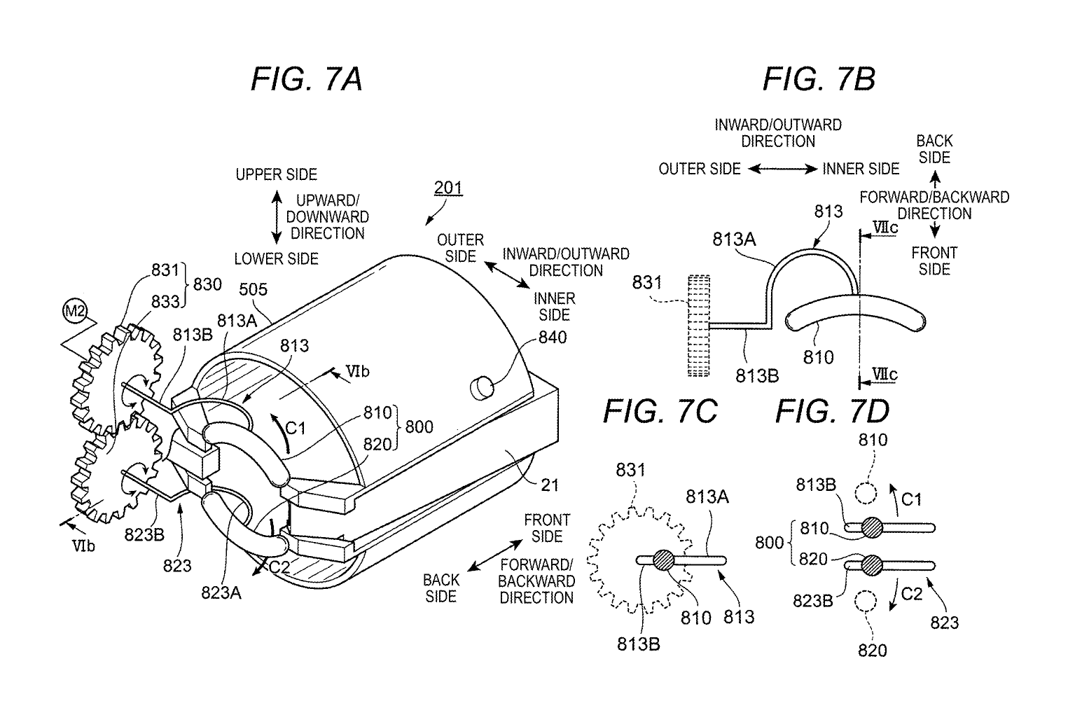

FIGS. 7A, 7B, 7C, and 7D are views describing a configuration of an optical measurement apparatus 201 in an alternative exemplary embodiment 2. Specifically, FIG. 7A is a perspective view of the optical measurement apparatus 201 when viewed from the back side, FIG. 7B is a top view of an upper eyelid pressing section 810 and a coupling member 813, FIG. 7C is a cross-sectional view taken along arrow VIIC in FIG. 7B, and FIG. 7D is a view describing an operation of an eyelid pressing section 800.

Subsequently, with reference to FIGS. 7A, 7B, 7C, and 7D, the alternative exemplary embodiment 2 will be described.

In the above-described optical measurement apparatus 101 illustrated in FIGS. 6A and 6B, description is given regarding the configuration in which the eyelid pressing section 700 is supported from the inner side of the holding section 500, for example, by the coupling members 713 and 723. However, the configuration is not limited thereto. For example, as in the optical measurement apparatus 101 illustrated in FIG. 7A, a configuration in which the eyelid pressing section 800 (upper eyelid pressing section 810 and lower eyelid pressing section 820) is supported from one side (outer side) in the inward/outward direction may be applied.

Specifically, the optical measurement apparatus 101 illustrated in FIG. 7A includes a motor M2, a gear group 830 (gears 831 and 833) that transmits driving force from the motor M2, coupling members 813 and 823 that are respectively connected to the upper eyelid pressing section 810 and the lower eyelid pressing section 820, and an operation button 840 that triggers driving of the motor M2. The motor M2, the gear group 830 (gears 831 and 833), and the coupling members 813 and 823 are provided by being supported by a holding section 505 (not illustrated).

Here, as illustrated in FIGS. 7B and 7C, the coupling member 813 is a so-called rod-like member having a substantially columnar shape. The coupling member 813 is provided along the inward/outward direction. The upper eyelid pressing section 810 is connected to an inner end portion thereof, and the gear 831 is connected to an outer end portion thereof. In addition, the coupling member 813 includes a curved portion 813A which is curved in the orientation of protruding to the back side in the forward/backward direction, and a linear portion 813B which leads to the outer end portion of the curved portion 813A. The linear portion 813B has the same axis as a rotary axis of the gear 831.

Similar to the coupling member 813, a coupling member 823 (detailed description will be omitted) is configured to include a curved portion 823A and a linear portion 823B.

An operation of the optical measurement apparatus 201 will be described.

First, the measurement subject wears the optical measurement apparatus 201 in the eyelids, and the upper eyelid pressing section 810 and the lower eyelid pressing section 820 come into contact with the upper eyelid 18 and the lower eyelid 19 (refer to FIG. 4B).

In this state, for example, when the measurement subject operates the operation button 840, the motor M2 is driven. In response to the driving of the motor M2, the coupling members 813 and 823 rotate via the gears 831 and 833. In accordance therewith, the upper eyelid pressing section 810 and the lower eyelid pressing section 820 are separated from each other, and the upper eyelid 18 and the lower eyelid 19 move in the orientation of being squeezed in the inward direction along the eyeball 10 (refer to arrows C1 and C2). Accordingly, the upper eyelid 18 and the lower eyelid 19 are open.

In this manner, clue to the configuration in which the upper eyelid pressing section 810 and the lower eyelid pressing section 820 are supported from one side in the inward/outward direction, a space inside the holding section 505 of the optical measurement apparatus 201 can be ensured.

### Alternative Exemplary Embodiment 3

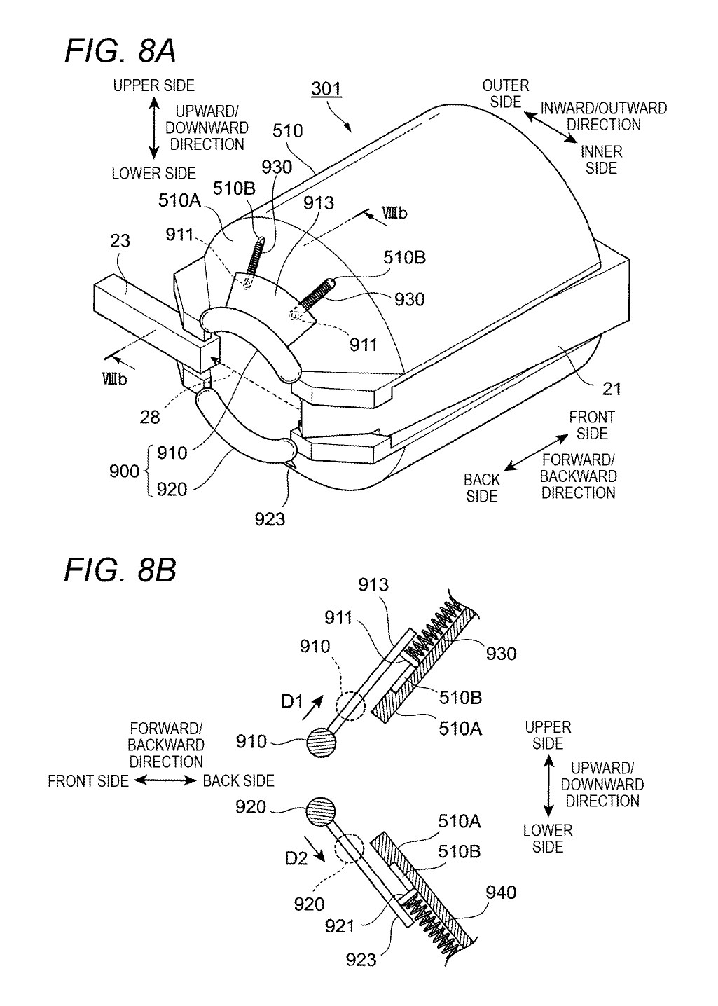

FIGS. 8A and 8B are views describing a configuration of an optical measurement apparatus 301 in an alternative exemplary embodiment 3. Specifically, FIG. 8A is a perspective view of the optical measurement apparatus 301 when viewed from the back side, and FIG. 8B is a cross-sectional view taken along arrow VIIIB in FIG. 8A.

Subsequently, with reference to FIGS. 8A and 8B, the alternative exemplary embodiment 3 will be described.

In FIGS. 6A, 6B, 7A, 7B, 7C, and 7D described above, description is given regarding the configuration in which the eyelid pressing sections 700 and 800 move by receiving driving force from the driving source. However, the configuration is not limited thereto. For example, as in the optical measurement apparatus 301 illustrated in FIG. 8A, a configuration in which an eyelid pressing section 900 (upper eyelid pressing section 910 and lower eyelid pressing section 920) moves by force of pressing the optical measurement apparatus 301 to the measurement subject may be applied.

Specifically, the optical measurement apparatus 301 illustrated in FIGS. 8A and 8B includes a truncated conical covering surface 510A which covers the back side of a holding section 510, and a guide groove 510B which is provided along the outer circumferential surface of the covering surface 510A and of which the longitudinal direction extends in the upward/downward direction. In addition, the optical measurement apparatus 301 includes pin-like guided portions 911 and 921 which are movably provided inside the guide groove 510B, a coupling member 913 which connects the guided portion 911 and the upper eyelid pressing section 910 together, a coupling member 923 which connects the guided portion 921 and the lower eyelid pressing section 920 together, and springs 930 and 940 which respectively bias the coupling members 913 and 923. Here, the springs 930 and 940 bias the coupling members 913 and 923 in the orientation in which the upper eyelid pressing section 910 and the lower eyelid pressing section 920 respectively connected to the coupling members 913 and 923 approach each other.

An operation of the optical measurement apparatus 301 will be described. First, the measurement subject wears the optical measurement apparatus 301 in the eyelids, and the upper eyelid pressing section 910 and the lower eyelid pressing section 920 come into contact with the upper eyelid 18 and the lower eyelid 19 (refer to FIG. 4B).

For example, when the measurement subject applies force of further pressing the optical measurement apparatus 301 to the upper eyelid 18 and the lower eyelid 19, the guided portions 911 and 921 of the coupling members 913 and 923 move inside the guide groove 510B against biasing force of the springs 930 and 940. Accordingly, the upper eyelid pressing section 910 and the lower eyelid pressing section 920 connected to the coupling members 913 and 923 move in the orientation of being separated from each other (refer to arrows D1 and D2). As a result thereof, the upper eyelid 18 and the lower eyelid 19 are open.

In this manner, in the optical measurement apparatus 301, without receiving driving force from the driving source, the measurement subject can reliably open the eyelids by utilizing the force of pressing the optical measurement apparatus 301 to the upper eyelid 18 and the lower eyelid 19.

### Modification Example

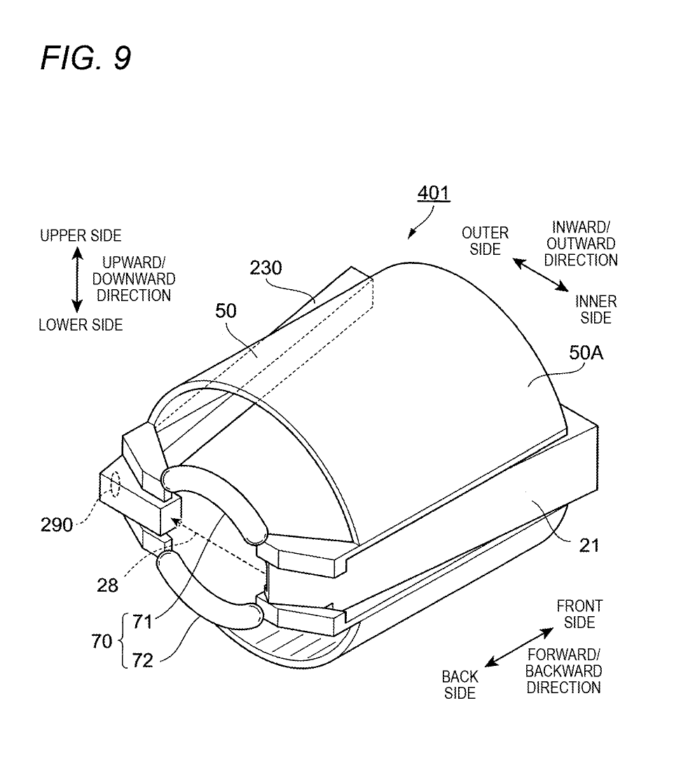

FIG. 9 is a view describing a configuration of an optical measurement apparatus 401 in a modification example.

In the description above, description is given regarding the configuration in which the light reception system 23 is disposed along the inward/outward direction. However, the configuration is not limited thereto.

For example, as in the optical measurement apparatus 401 illustrated in FIG. 9, a configuration in which a light reception system 230 is provided along the forward/backward direction may be applied. For example, the light reception system 230 includes a mirror 290 which is provided at the end portion of the light reception system 230 on the back side, receives light which has passed through the anterior chamber 13 (refer to FIG. 2), and reflects the light toward the front side in the forward/backward direction.

As in the optical measurement apparatus 401, when the light reception system 230 is provided along the forward/backward direction, the size in the inward/outward direction may be restrained. In addition, for example, the optical measurement apparatus 401 can also be used for any of the right and left eyeballs 10 (refer to FIG. 2).

In the description above, description is given regarding the configuration in which the eyelid pressing section 70 includes plural members (upper eyelid pressing section 71 and lower eyelid pressing section 72). However, the configuration is not limited thereto. For example, the eyelid pressing section 70 may be configured with any one of the upper eyelid pressing section 71 and the lower eyelid pressing section 72. Otherwise, a configuration in which the upper eyelid pressing section 71 and the lower eyelid pressing section 72 are integrally formed may be applied.

In addition, the upper eyelid pressing section 71 and the lower eyelid pressing section 72 may have a shape different from that described above. For example, the upper eyelid pressing section 71 and the lower eyelid pressing section 72 may not be curved. Specifically, as long as the upper eyelid pressing section 71 and the lower eyelid pressing section 72 come into contact with at least any of the upper eyelid 18 and the lower eyelid 19 (refer to FIG. 4B) and a state where a gap to the extent that light can pass through between the upper eyelid 18 and the lower eyelid 19 is formed may be maintained, a different shape such as a spherical member, a plate-shaped member, and the like may also be applied naturally.

In addition, any one of the upper eyelid pressing section 71 and the lower eyelid pressing section 72 may be movably configured as described in FIGS. 6A, 6B, 7A, 7B, 7C, 7D, 8A, and 8B, and the other one may be fixedly configured as described in FIG. 1 and the like. For example, a configuration in which the upper eyelid pressing section 71 is movable and the lower eyelid pressing section 72 is fixed may be applied.

In addition, description is given regarding the configuration in which the eyelid pressing section 70 (upper eyelid pressing section 71 and lower eyelid pressing section 72) presses the eyelids of the measurement subject. However, as long as the eyelid pressing section 70 comes into contact with the skin or the like on the periphery of the eyeball 10 of the measurement subject and the eyelids of the measurement subject are maintained in an open state, the configuration is not limited thereto.

The periphery of the eyeball 10 of the measurement subject denotes a region within a range in which when the eyelid pressing section 70 comes into contact, movement (opening and closing) of at least any one of the upper eyelid 18 and the lower eyelid 19 is restricted to a great or small extent. In addition, for example, sites on the periphery of the eyeball 10 of the measurement subject other than the eyelids include an eyebrow 26 (refer to FIG. 4B), the temple, the cheek, and the like of the measurement subject.

In addition, in the description above, “restricting movement of the eyelids” and “maintaining the eyelids in an open state” denote a state where movement of the eyelids in a direction of being closed is restricted to a great or small extent, compared to a state where nothing comes into contact with the site on the periphery of the eyeball, such as the eyelids, and does not denote a state of being completely restricted or maintained. As long as a configuration in which restricting or maintaining thereof is performed with stress such that the optical path is not blocked due to a blinking motion of the measurement subject, a measurement may be performed without being influenced by the blinking motion.

In addition, in the description above, description is given regarding the method of calculating the concentration of the intended optically active substance contained in the aqueous humor. However, a configuration in which different characteristics of the aqueous humor are measured may be applied.

In addition, not only the characteristics related to the aqueous humor, a configuration described in the present exemplary embodiment in order to obtain the characteristics related to the cornea or the like which is present in the optical path 28 may be applied. That is, as long as light is incident from the outside of the eyeball 10, the light passes through the aqueous humor in the cornea 14 and the anterior chamber 13, and the characteristics related to the eyeball 10 are obtained, the configuration described in the present exemplary embodiment may be applied.

In addition, in description of the present exemplary embodiment, description is given regarding a case of the eyeball 10 of the left eye. However, the optical measurement apparatus 1 may be applied to the eyeball of the right eye (not illustrated).

In addition, in the description above, the method in which the measurement subject holds the optical measurement apparatus in a hand and wears the optical measurement apparatus by himself/herself is described. However, an assistant or the like may hold the optical measurement apparatus in a hand and put the optical measurement apparatus with respect to the measurement subject. In addition, in a state where the optical measurement apparatus is fixed, the measurement subject may wear the optical measurement apparatus by moving his/her facial surface toward the optical measurement apparatus. Moreover, by using a driving source which can move the optical measurement apparatus in the forward/backward direction of the measurement subject, the measurement subject may wear the optical measurement apparatus by using the driving source in a state where the position of the facial surface is fixed.

In addition, in the description above, description is given regarding the configuration in which the light emission system, the light reception system, and the eyelid pressing section are integrated in the holding section. However, the members are not necessarily configured to be integrated.

In the description above, description is given regarding the configuration in which in a case where the eyelid pressing section is driven, the motor is used as the driving source and the power thereof is transmitted to the eyelid pressing section via the transmitting portion such as the gear and the coupling member, or the support portion. However, a configuration in which no motor is used and the eyelid pressing section is manually open and closed via the transmitting portion or the support portion may be applied. Moreover, a configuration in which no transmitting portion is used and the eyelid pressing section is manually and directly open and closed may be applied.

In addition, various types of exemplary embodiments and the modification example are described above. However, naturally, a configuration in which the exemplary embodiments and the modification example are combined together may also be applied.

In addition, the present disclosure is not limited by the exemplary embodiments at all and may be executed in various types of forms without departing from the scope and the gist of the present disclosure.

What is claimed is:

1. A measurement apparatus comprising:
an eyelid opening section that comes into contact with skin on a periphery of an eyeball of a measurement subject and maintains an eyelid of the measurement subject in an open state;
an emission section that emits light such that the light travels across an anterior chamber of the eyeball with the eyelid maintained in an open state by the eyelid opening portion; and
a light reception section that receives the light which travels across the anterior chamber.

2. The measurement apparatus according to claim 1, further comprising:
a holding section that holds the eyelid opening section, the emission section, and the light reception section at a position where the light emitted from the emission section travels across the anterior chamber of the eyeball with the eyelid maintained in an open state by the eyelid opening section and the light is received by the light reception section.

3. A measurement apparatus comprising:
an emission section that emits light such that the light travels across an anterior chamber of an eyeball of a measurement subject;
a light reception section that receives the light which travels across the anterior chamber; and
an eyelid opening section that, when the emission section and the light reception section are positioned with respect to the eyeball, opens an eyelid of the eyeball by squeezing the eyelid in an inward direction with respect to the eyeball in response to movement of the emission section and the light reception section in the inward direction with respect to the eyeball.

4. A measurement apparatus comprising:
an emission section that emits light such that the light travels across an anterior chamber of an eyeball of a measurement subject;
a light reception section that receives the light which travels across the anterior chamber; and
an eyelid opening section that is provided at a position where the eyelid opening section comes into contact with skin on a periphery of the eyeball in accordance with a positioning operation of the emission section and the light reception section performed with respect to the eyeball, the position being a position where an eyelid opening section can open an eyelid in a state where the emission section and the light reception section are positioned.

5. The measurement apparatus according to claim 1, wherein
the eyelid opening section includes an upper eyelid pressing portion that presses an upper eyelid of the measurement subject and a lower eyelid pressing portion that presses a lower eyelid of the measurement subject.

6. The measurement apparatus according to claim 3, wherein
the eyelid opening section includes an upper eyelid pressing portion that presses an upper eyelid of the measurement subject and a lower eyelid pressing portion that presses a lower eyelid of the measurement subject.

7. The measurement apparatus according to claim 4, wherein
the eyelid opening section includes an upper eyelid pressing portion that presses an upper eyelid of the measurement subject and a lower eyelid pressing portion that presses a lower eyelid of the measurement subject.

8. The measurement apparatus according to claim 1, wherein
one of the emission section and the light reception section is disposed on an inner canthus side and other of the emission section and the light reception section is disposed on an outer canthus side.

9. The measurement apparatus according to claim 3, wherein
one of the emission section and the light reception section is disposed on an inner canthus side and other of the emission section and the light reception section is disposed on an outer canthus side.

10. The measurement apparatus according to claim 4, wherein
one of the emission section and the light reception section is disposed on an inner canthus side and other of the emission section and the light reception section is disposed on an outer canthus side.

11. The measurement apparatus according to claim 1, further comprising:
a support section that supports the eyelid opening section movably in a direction in which the eyelid opens.

12. The measurement apparatus according to claim 3, further comprising:
a support section that supports the eyelid opening section movably in a direction in which the eyelid opens.

13. The measurement apparatus according to claim 4, further comprising:
a support section that supports the eyelid opening section movably in a direction in which the eyelid opens.

14. The measurement apparatus according to claim 11, further comprising:
a driving source; and
a transmitting section that transmits driving force from the driving source to the support section and moves the support section in the direction in which the eyelid opens.

12. The measurement apparatus according to claim 12, further comprising:
a driving source; and
a transmitting section that transmits driving force from the driving source to the support section and moves the support section in the direction in which the eyelid opens.

16. The measurement apparatus according to claim 13, further comprising:
a driving source; and
a transmitting section that transmits driving force from the driving source to the support section and moves the support section in the direction in which the eyelid opens.

17. A measurement apparatus comprising:
an emission section that emits light such that the light travels across an anterior chamber of an eyeball of a measurement subject;
a light reception section that receives the light which travels across the anterior chamber; and
a restriction section that, when the emission section and the light reception section are positioned with respect to the eyeball, comes into contact with skin on a periphery of the eyeball in response to movement of the emission section and the light reception section in an inward direction with respect to the eyeball, and restricts movement of an eyelid in a direction of closing.

18. The measurement apparatus according to claim 17. wherein
the restriction section comes into contact with the eyelid of the eyeball and restricts movement of the eyelid in the direction of closing.
